(19) United States
(12) Patent Application Publication
(10) Pub. No.: US 2008/0281340 A1
(43) Pub. Date: Nov. 13, 2008

(54) Surgical cutting device

(75) Inventors: Sinjae Hyun, Macon, GA (US); Amanda Elizabeth Chappell, Tullahoma, TN (US); Jake Lee Dickson, Toccoa, GA (US); Clement Kleinstreuer, Raleigh, NC (US); Christopher Allen Walters, Hartselle, AL (US)

Correspondence Address: WITHERS & KEYS, LLC, P. O. BOX 2049, MCDONOUGH, GA, 30253, US

(73) Assignee: Mercer University

(21) Appl. No.: 11/801,354

(22) Filed: May 9, 2007

(51) Int. Cl. A61B 17/122

(52) U.S. Cl. 606/157; 606/184

(57) ABSTRACT

A surgical cutting device that clamps and cuts a graft creating optimal geometry for end-to-end anastomosis is disclosed. Methods of making and using the surgical cutting device are also disclosed.

### TECHNICAL FIELD

The present invention is directed to surgical cutting devices and methods of making and using surgical cutting devices.

### BACKGROUND

Anastomosis is a vascular surgical procedure that reconstructs an occluded, or blocked, thigh artery in order to restore blood circulation and prevent complications that could result in a heart attack and/or limb amputation. This surgical procedure is performed when the buildup of fatty deposits (i.e., plaque) in an artery block the normal flow of blood that carries oxygen and nutrients to the lower extremities. A common method of reconstruction is to bypass the occluded portion of the artery with a prosthetic arterio-venous graft that is connected to the artery. Typically, the prosthetic graft is either a segment of the autogenous saphenous vein, a vein that runs the length of the leg, or a synthetic graft typically formed from polytetrafluoroethylene (PTFE) material. The graft is connected to two ends of the artery, the proximal and distal ends, in order to bypass the blockage within the artery. The proximal end of the artery is located before the occluded area where the blood flow becomes restricted. The distal end is located after the blockage.

The surgical procedure of end-to-side anastomosis is specifically employed to the distal end of the artery allowing blood flow from the prosthetic graft back into the artery. This procedure is routinely used in anastomosis procedures such as in the femoral popliteal artery, an artery found in the thigh. The most commonly used prosthetic graft for femoral popliteal end-to-side anastomosis is the saphenous vein, but when this vein is not available, a surgeon will use a PTFE synthetic material graft.

In a typical bypass surgery, a prosthetic graft is sewn to the artery to allow blood flow around a blocked artery. Despite improvements in medical technology, as well as operation techniques, many patients find themselves undergoing the same operation every four to five years. These frequent operations cause more long-term damage to the artery and create unnecessary stress on the patient's cardiovascular system. If the procedure can be improved to decrease the need for subsequent operations, patients would benefit tremendously.

Multiple procedures are used to prepare the graft prior to connection to the artery in end-to-side anastomosis, but all procedures require the prosthetic graft to be cut before connection. In addition to other techniques, the graft can be cut at an angle with shears along an applied clamp or a U-shaped slit can be cut into the graft for use with a vein patch. The prepared graft is then attached to the occluded artery. FIG. 1 shows a detailed picture of the connection between a prosthetic graft and an occluded artery. As shown in FIG. 1, a graft 1 is connected to an artery 2 in order to create an arterial junction 3. Arterial junction 3 comprises an inlet 4, a heel 5, a toe 6, and an arterial floor 7. Artery 2 requires an incision (i.e., a slit or cut) along the length of artery 2 that is approximately 1.5 to 2 times longer than the diameter of graft 1. After the incision is applied to artery 2, graft 1 is sutured (i.e., stitched) to artery 2. This connection creates arterial junction 3 that reroutes the blood flow past an occluded segment (not shown) of artery 2. The angle between artery 2 and graft 1 is the anastomosis attachment angle 8.

During the surgical process of end-to-side anastomosis, the surgeon must make certain decisions including, but not limited to, what angle graft 1 is to be cut so as to produce a desired anastomosis attachment angle 8. For example, arterial junction 3 created by graft 1 and artery 2 can have several variables, such as the angle at which the graft 1 is attached and the design of the graft cut (e.g., a straight cut, a curved cut, an S-shaped cut, etc.). Typically, graft 1 is attached so as to form an attachment angle 8 of between about 100 and about 70°, more typically, between about 30° and about 55°. FIG. 2 shows a connection between artery 9 and graft 10 with attachment angle 8 equal to about 30°.

A significant problem with end-to-side anastomosis is graft failure at the site of connection. Graft failure may be created by intimal hyperplasia (IH) development, the human body's response to an injury that creates scar tissue in order to repair the site of the injury. This scar tissue continually builds at the site of injury, causing the new arterial junction to become occluded. For the femoral anastomotic case, scar tissue most often forms at the site of the suture on the toe region and at the arterial floor where the blood flow reenters the artery. (See, for example, FIG. 1.) This intimal thickening at the graft junction accounts for the major source of graft failures.

The current patency life on a prosthetic graft surgically attached using an end-to-side anastomosis surgical procedure is approximately five years. Many of these grafts do not last the expected life (i.e., last less than five years, and in some cases last less than 3 years). See, for example, Sachez, L. A. et al., J Vascular Surgery, 1993, Vol. 18, pp. 981-989, the content of which is incorporated herein by reference in its entirety. These prosthetic grafts often experience failure due to IH development that will eventually cause the new artery to become occluded and often leading to additional surgeries.

There exists a need in the art for a clamping and cutting device operatively adapted to cut synthetic grafts suitable for use in an end-to-side anastomosis surgical procedure. Further, there exists a need in the art for cut synthetic grafts that can potentially increase the expected life of the graft when used in an end-to-side anastomosis surgical procedure by effectively reducing IH development at the distal arterial graft junction.

### SUMMARY

The present invention is directed to a hand-held clamping and cutting device operatively adapted to cut objects such as synthetic grafts. In one exemplary embodiment, the hand-held clamping and cutting device comprises an upper clamping member having an upper surface and an upper clamping surface, and an opening extending through the upper clamping member from the upper surface to the upper clamping surface; a lower clamping member having an lower clamping surface, wherein the upper and lower clamping members are operatively adapted to provide a clamping pressure on an object disposed therebetween; a blade housing positioned above the upper clamping member; and a blade connected to the blade housing; wherein the blade housing is operatively adapted to move toward the upper surface so that the blade extends through the opening and contacts the lower clamping surface. The exemplary hand-held clamping and cutting device may comprise a number of additional features including, but not limited to, a mechanism for moving the upper clamping surface relative to the lower clamping surface, alignment members for aligning an object relative to the lower clamping surface and/or upper clamping surface, and one or more blades for use in the blade housing.

The present invention is also directed to a kit comprising a hand-held clamping and cutting device, and a set of blades having two or more blade configurations. In this exemplary embodiment of the present invention, a given blade housing may be used with two or more blades having different blade configurations (e.g., straight, curved, etc.). In other embodiments of the present invention, an exemplary kit may further comprise a set of blade housings that are operatively adapted to be used with a single blade having a unique blade configuration that corresponding to a similarly unique blade housing configuration. In other embodiments of the present invention, the exemplary kit may further comprise one or more synthetic grafts, such as a set of two or more synthetic grafts, wherein each graft has a unique set of graft dimensions which differ from other grafts within the set.

The present invention is further directed to methods of making hand-held clamping and cutting devices. In one exemplary embodiment of the present invention, the method of making a hand-held clamping and cutting device comprises providing an upper clamping member having an upper surface and an upper clamping surface, and an opening extending through the upper clamping member from the upper surface to the upper clamping surface; providing a lower clamping member having an lower clamping surface; connecting the lower clamping member to the upper clamping member so that the upper and lower clamping surfaces face one another and are operatively adapted to provide a clamping pressure on an object disposed therebetween; providing a blade housing positioned above the upper clamping member, wherein the blade housing comprises a blade dimensioned so as to extends through the opening, the blade housing being operatively adapted to move toward the upper surface so that the blade extends through the opening and approaches the lower clamping surface.

The present invention is even further directed to methods of using hand-held clamping and cutting devices. In one exemplary embodiment of the present invention, the method of using a hand-held clamping and cutting device comprises a method of cutting an object, the exemplary method comprising (A) placing the object between upper and lower clamping surfaces of a clamping and cutting device, the clamping and cutting device comprising (i) an upper clamping member having an upper surface and the upper clamping surface, and an opening extending through the upper clamping member from the upper surface to the upper clamping surface; (ii) a lower clamping member having the lower clamping surface, wherein the upper and lower clamping members are operatively adapted to provide a clamping pressure on the object; (iii) a blade housing positioned above the upper clamping member; and (iv) a blade connected to the blade housing, wherein the blade housing is operatively adapted to move toward the upper surface so that the blade extends through the opening and approaches the lower clamping surface; and (B) moving the blade housing toward the object so that the blade contacts and cuts the object. The exemplary method of cutting an object is particularly useful for cutting grafts in preparation for an anastomosis surgical procedure.

Through the use of the hand-held clamping and cutting device of the present invention and computer-aided fluid flow models, a specific blade geometry has been discovered that creates a graft having an end cut that, when employed in an anastomosis surgical procedure, provides enhanced blood flow through the graft. The hand-held clamping and cutting device of the present invention is able to produce a cut on a graft that reduces IH by decreasing abnormal hemodynamic parameters at the reconstructed arterial junction.

These and other features and advantages of the present invention will become apparent after a review of the following detailed description of the disclosed embodiments and the appended claims.

### BRIEF DESCRIPTION OF THE DRAWINGS

The present invention may be readily understood by the following detailed description in conjunction with the accompanying drawings, wherein:

FIG. 1 depicts an arterial junction;

FIG. 2 depicts an artery and graft attached at an anastomosis attachment angle of 30°;

FIG. 3 depicts a frontal view of an exemplary hand-held clamping and cutting device of the present invention;

FIG. 4 depicts a side view of another exemplary hand-held clamping and cutting device of the present invention;

FIG. 5 depicts an exemplary lower handle suitable for use in the exemplary hand-held clamping and cutting device shown in FIG. 4;

FIG. 6 depicts an exemplary upper handle suitable for use in the exemplary hand-held clamping and cutting device shown in FIG. 4;

FIG. 7 depicts the exemplary lower handle of FIG. 5 and the exemplary upper handle of FIG. 6 positioned relative to one another;

FIG. 8 depicts an exemplary blade housing suitable for use in the exemplary hand-held clamping and cutting device shown in FIG. 4 in combination with the exemplary lower handle of FIG. 5 and the exemplary upper handle of FIG. 6;

FIG. 9 depicts a view of the exemplary blade housing shown in FIG. 8 as viewed from below the blade housing;

FIG. 10 depicts an exemplary blade housing and blade assembly suitable for use in the exemplary hand-held clamping and cutting device shown in FIG. 4;

FIG. 11 depicts an exemplary blade holder and blade suitable for use in the exemplary hand-held clamping and cutting device shown in FIG. 4 when viewed from below the blade holder;

FIG. 12 depicts the result of cutting a tubular object using the exemplary hand-held clamping and cutting device shown in FIG. 4;

FIG. 13 graphically depicts simulated particle flow resident time through an occlusion; and

FIGS. 14a-14h depict exemplary process steps of cutting a synthetic graft using the exemplary hand-held clamping and cutting device shown in FIG. 4.

### DETAILED DESCRIPTION

The present invention is directed to hand-held clamping and cutting devices operatively adapted to cut objects such as synthetic grafts. The present invention is also directed to kits comprising one or more hand-held clamping and cutting devices, an optional set of blades having two or more blade configurations, and an optional set of grafts having two or more graft configurations. The present invention is even further directed to methods of using hand-held clamping and cutting devices such as a method of cutting an object such as a synthetic graft.

A number of terms are used to describe the disclosed hand-held clamping and cutting devices and methods of using the hand-held clamping and cutting devices of the present invention. Some of these terms are described below.

As used herein, the term “graft” refers to any tubular object including, but not limited to, synthetic grafts, biografts or biosynthetic grafts. Exemplary grafts include, but are not limited to, polytetrafluoroethylene (PTFE) grafts commercially available from Atrium Medical Corporation (Hudson, N.H.). The cross-sectional structure of a given graft is typically circular, but may be any tubular shape including, but not limited to, triangular, polygonal, square, rectangular, etc.

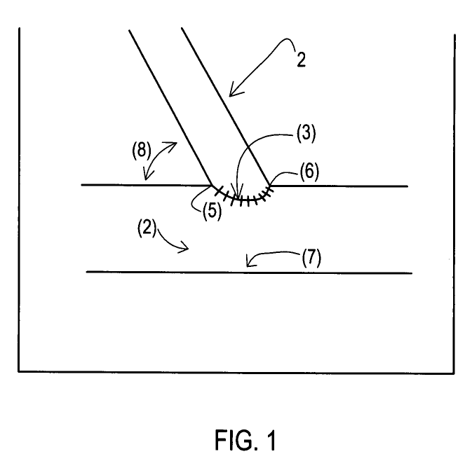

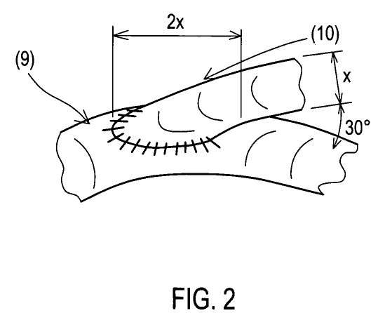

As used herein, the term “anastomosis attachment angle” is the angle at which a given graft attaches to an artery. This angle is also referred to herein as the “attachment angle” or the “anastomosis angle.” See, for example, FIG. 1.

As used herein, the term “proximal” is used to refer to an end of the disclosed hand-held clamping and cutting device which is closest to an operator, while the term “distal” is used to refer to an end of the hand-held clamping and cutting device which is furthest from the operator.

### I. Clamping and Cutting Devices

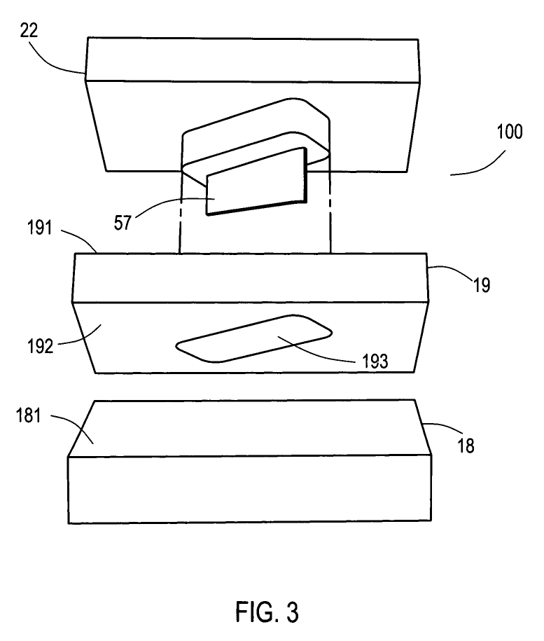

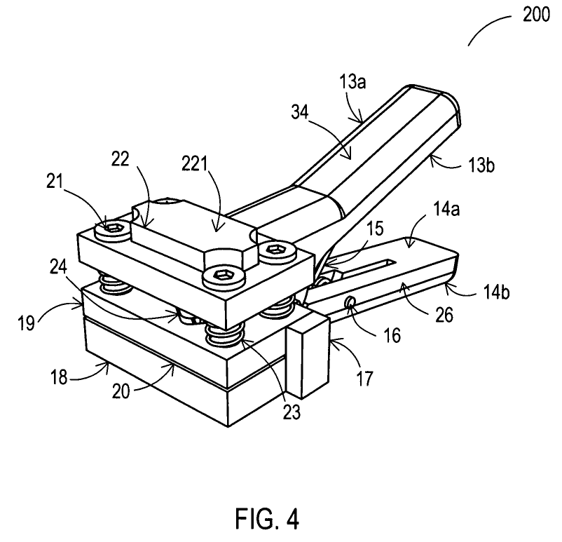

The present invention is directed to hand-held clamping and cutting devices operatively adapted to cut objects such as synthetic grafts. One exemplary hand-held clamping and cutting device of the present invention is depicted in FIG. 3. As shown in FIG. 3, exemplary hand-held clamping and cutting device 100 comprises an upper clamping member 19 having an upper surface 191 and an upper clamping surface 192, and an opening 193 extending through upper clamping member 19 from upper surface 191 to upper clamping surface 192; a lower clamping member 18 having an lower clamping surface 181, wherein upper and lower clamping members 19,18 are operatively adapted to provide a clamping pressure on an object (not shown) disposed therebetween; a blade housing 22 positioned above upper clamping member 19; and a blade 57 connected to blade housing 22; wherein blade housing 22 is operatively adapted to move toward upper surface 191 so that blade 57 extends through opening 193 and contacts lower clamping surface 181. Desirably, upper and lower clamping surfaces 192,181 are substantially planar, and when exemplary hand-held clamping and cutting device 100 is at rest, upper and lower clamping surfaces 192,181 are substantially parallel with one another. As described below, exemplary hand-held clamping and cutting device 100 may comprise a number of additional features including, but not limited to, a mechanism for moving upper clamping surface 192 relative to lower clamping surface 181, alignment members 17 (see, FIG. 4) for aligning an object (not shown) relative to lower clamping surface 181 and/or upper clamping surface 192, and one or more blades 57 for use in blade housing 22.

The hand-held clamping and cutting device of the present invention may further comprise upper and lower handles, wherein the upper and lower clamping members are connected to upper and lower handles respectively, and the upper and lower handles are operatively adapted to move the upper clamping surface relative to the lower clamping surface. In one exemplary embodiment, the upper and lower handles are connected to one another such that when the upper and lower handles are moved toward one another, the upper clamping surface moves away from the lower clamping surface. Such an exemplary device is depicted in FIG. 4.

FIG. 4 provides a side view of an exemplary hand-held clamping and cutting device of the present invention comprising upper and lower handles. As shown in FIG. 4, exemplary hand-held clamping and cutting device 200 comprises upper clamping member 19; lower clamping member 18; an upper handle 13 located at a distal end of exemplary hand-held clamping and cutting device 200 and having a top surface 13a and a lower surface 13b; and lower handle 14 having a top surface 14a and a lower surface 14b. Upper handle 13 and lower handle 14 are separated and held apart by torsion spring 15, which is secured to lower handle 14 via screw 16. During operation of exemplary hand-held clamping and cutting device 200, torsion spring 15 is of sufficient strength to hold the distal portion of upper handle 13 in a spaced apart relationship from the distal end of lower handle 14 while maintaining upper clamping member 19 in substantial contact with or spaced from lower clamping member 18. Torsion spring 15 is operatively adapted to resist movement of upper and lower handles 13,14 toward one another. Further, torsion spring 15 is operatively adapted so that an operator of exemplary hand-held clamping and cutting device 200 can squeeze upper and lower handles 13,14 toward one another.

During operation of exemplary hand-held clamping and cutting device 200, pressure is applied to upper handle 13 and/or lower handle 14, forcing upper and lower handles 13,14 closer together, while simultaneously separating lower clamping member 18 from upper clamping member 19. When no force is being applied to either upper handle 13 or lower handle 14, upper clamping member 19 and lower clamping member 18 are desirably separated by a gap 20 of a predetermined size, depending on the dimensions of the object (not shown) to be cut (e.g., the diameter of a graft). In one exemplary embodiment, gap 20 typically has a gap thickness (i.e., the distance between upper clamping surface 192 and lower clamping surface 181) of up to about 1 cm, more typically, up to about 0.5 cm.

When upper handle 13 and lower handle 14 are forced toward one another and upper and lower clamping members 19,18 are separated, an object to be cut (e.g., a graft) is desirably positioned between upper and lower clamping members 19,18 such that a longitudinal axis of the object is essentially perpendicular to a longitudinal axis of exemplary hand-held clamping and cutting device 200 extending along upper and lower handles 13,14. The object to be cut (e.g., a graft) is positioned against optional alignment members 17 positioned along opposite sides of lower clamping member 18, upper clamping member 19, or both upper and lower clamping members 19,18. By releasing the pressure on upper and lower handles 13,14 of exemplary hand-held clamping and cutting device 200, upper and lower clamping members 19,18 move toward one another and provide a clamping pressure on an object positioned therebetween within gap 20.

Desirably, exemplary hand-held clamping and cutting device 200 comprises one or more alignment members 17 extending along at least lower clamping member 18, the one or more alignment members 17 being operatively adapted to align an object (not shown) on lower clamping surface 181 in a first direction. Typically, when present, the one or more alignment members 17 are present as a pair of alignment members 17 extending upward along opposite sides of lower clamping member 18 toward upper clamping member 19.

To cut an object using exemplary hand-held clamping and cutting device 200, pressure is applied onto blade housing 22 so as to force blade housing 22 with blade 57 toward the object positioned within gap 20. As shown in FIG. 4, in exemplary hand-held clamping and cutting device 200, blade housing 22 is positioned above and attached to upper clamping member 19 by four shoulder screws 21. Compression springs 23 positioned along shoulder screws 21 provide tension so as to separate (i) blade housing 22 from upper clamping member 19 and (ii) blade 57 from the object to be cut. By applying pressure onto an upper surface 221 of blade housing 22, blade 57 moves toward the object positioned within gap 20, passing through opening 193 in upper clamping member 19, so as to cut the object. Blade 57 may contact upper clamping surface 181 of lower clamping member 18 or stop just prior to touching upper clamping surface 181 of lower clamping member 18.

Desirably, blade housing 22 is separated from upper surface 191 of upper clamping member 19 by at least one compression spring 23, such that upon applying suitable pressure to blade housing 22, the at least one compression spring 23 will depress and blade 57 will pass through opening 193 and contact and/or extend into lower clamping surface 181. In one desired embodiment (shown in FIG. 4), blade housing 22 is separated from upper surface 191 of upper clamping member 19 by four compression springs 23 positioned along an outer periphery of upper clamping member 19.

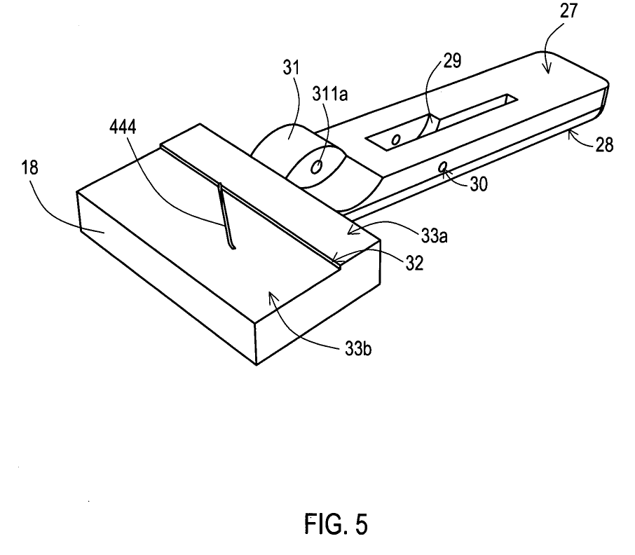

FIG. 5 provides a detailed view of exemplary lower handle 26 of exemplary hand-held clamping and cutting device 200 shown in FIG. 4. As shown in FIG. 5, exemplary lower handle 26 comprises a lower handle top surface 27, a lower handle bottom surface 28, and a recess 29 extending along and within lower handle top surface 27 for housing torsion spring 15 (not shown) described above. Exemplary lower handle 26 further comprises attachment member 31 having hole 311 extending there through. Attachment member 31 attaches to a complimentary upper handle portion (e.g., attachment member 312 of upper handle 34) described below. Lower clamping member 18 of exemplary lower handle 26 comprises a raised surface part 33a and a recessed surface part 33b. The distance separating raised surface part 33a and recessed surface part 33b is defined by a ridge 32. The height of ridge 32 is chosen to provide a desired width of gap 20, which varies depending on the dimensions of the object to be cut (i.e., the diameter of a synthetic graft to be cut).

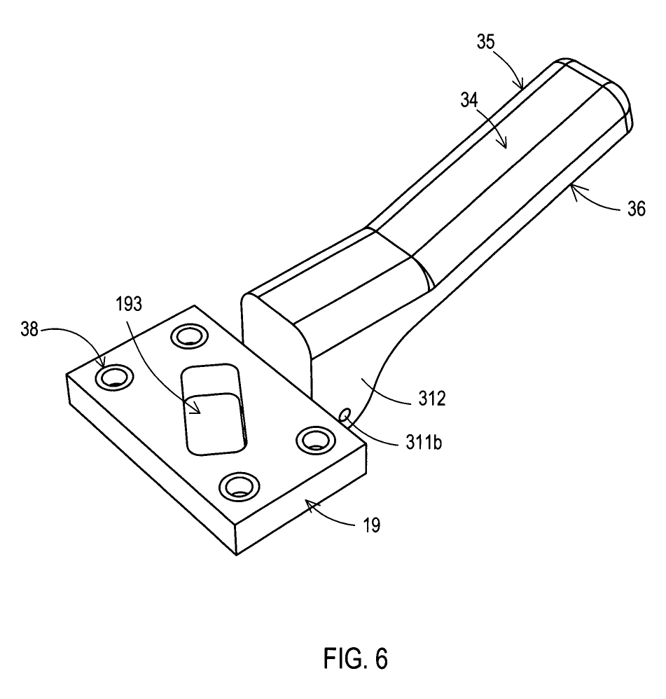

FIG. 6 provides a detailed view of exemplary upper handle 34 of exemplary hand-held clamping and cutting device 200 shown in FIG. 4. As shown in FIG. 6, exemplary upper handle 34 comprises an upper handle top surface 35, an upper handle lower surface 36, and attachment member 312 for attaching to attachment member 31 of lower handle 26 via hole 311 (e.g., a pin (not shown) extending through hole 311). Upper clamping member 19 of exemplary upper handle 34 comprises opening 193 extending there through. Although opening 193 is shown as having an oval shape, it should be understood that opening 193 may have any opening shape depending on the desired position, shape and size of blade housing 22 and blade 57. Upper clamping member 19 of exemplary upper handle 34 further comprises shoulder screw holes 38 for housing shoulder screws 21 (shown in FIG. 4), which optionally connect blade housing 22 (shown in FIGS. 3-4) to upper clamping member 19.

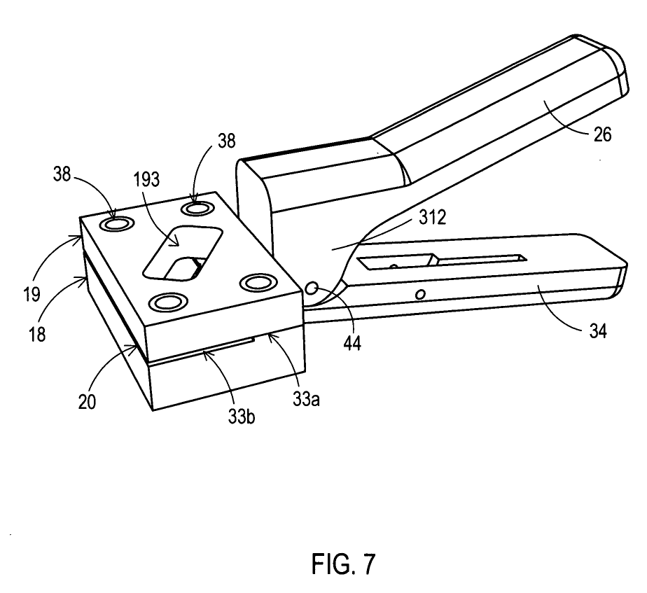

FIG. 7 provides a view of exemplary lower handle 26 of FIG. 5 and exemplary upper handle 34 of FIG. 6 positioned relative to one another. Exemplary lower handle 26 and exemplary upper handle 34 pivot relative to one another along pivot 44, which extends through holes 311a and 311b of attachment members 31 and 312 respectively (see, FIGS. 5-6). As shown in FIG. 7, when exemplary hand-held clamping and cutting device 200 is at rest, lower clamping member 18 is in substantial contact with upper clamping member 19 such that raised surface 33a of lower clamping member 18 is in intimate contact with upper clamping surface 192 of upper clamping member 19 and recessed surface 33b of lower clamping member 18 is separated from upper clamping surface 192 of upper clamping member 19 by a predetermined distance representing a width of gap 20. As discussed above, blade housing 22 and blade 57 may be positioned above and extend into opening 193.

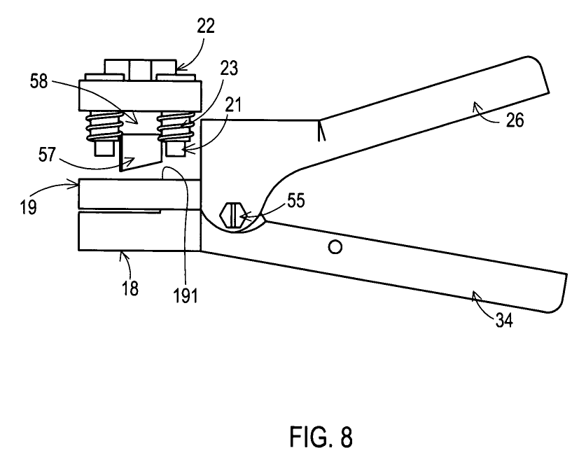

FIG. 8 provides a view of an exemplary blade housing suitable for use in the exemplary hand-held clamping and cutting device shown in FIG. 4 in combination with exemplary lower handle 26 of FIG. 5 and exemplary upper handle 34 of FIG. 6. As shown in FIG. 8, exemplary blade housing 22 comprises a blade holder 58 and blade 57. Blade housing 22 is optionally connected to upper surface 191 of upper clamping member 19 via shoulder screws 21, and may be moved toward upper surface 191 of upper clamping member 19 by applying force to compression springs 23. Upper handle 34 and lower handle 26 pivot around pivot screw 55.

As shown in FIG. 8, lower clamping member 18 is integrally connected to lower handle 34, while upper clamping member 19 is integrally connected to upper handle 26. Although lower clamping member 18 and lower handle 34 are shown as a single piece and upper clamping member 19 and upper handle 26 are shown as a single piece, it should be noted that other configurations are contemplated by the present invention including embodiments in which lower clamping member 18, lower handle 34, upper clamping member 19 and upper handle 26 are each separate pieces joined so as to form a clamping device. In other alternative embodiments, lower clamping member 18 is integrally connected to upper handle 26, while upper clamping member 19 is integrally connected to lower handle 34 so as to form a device having a pivot point similar to a pair of scissors.

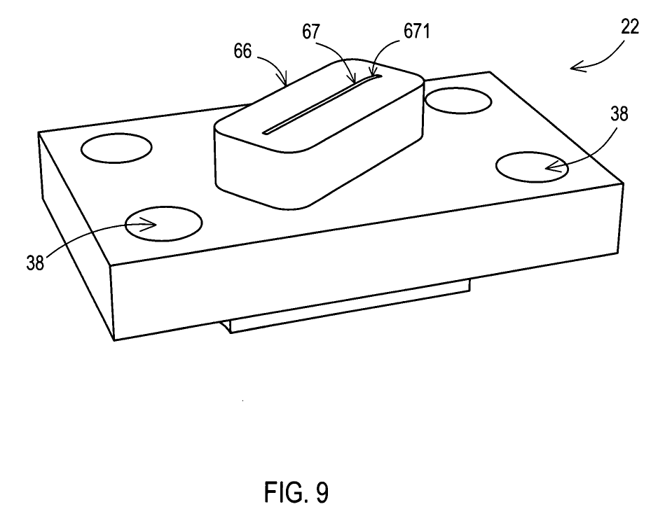

FIG. 9 provides a detailed view of exemplary blade housing 22 as viewed from below exemplary blade housing 22. Blade housing 22 comprises a blade holder 66 having a blade slot 67 therein for housing a blade (not shown). As shown in this exemplary embodiment, the end 671 of blade slot 67 is curved to accommodate a curved blade in accordance with a preferred embodiment of the present invention. Blade holder 66 may have any shape and may be removable from blade housing 22 or attached to blade housing 22 via lateral screws (not shown) or any other hardware or glue. In an alternative embodiment, blade housing 22 and blade holder 67 may be one piece, such as a molded plastic or cast metal part.

Holes 38 may be used to optionally attach blade housing 22 to the upper clamping member (not shown) as described above. Any means of attaching blade housing 22 to the upper clamping member is suitable so long as blade housing 22 and/or blade holder 66 and blade 57 are movable toward and away from an object to be cut. As described above, in one exemplary embodiment, compression springs 23 and shoulder screws 22 are used to connect blade housing 22 to upper clamping member 19 such that blade housing 22 can move toward upper clamping member 19 and an object to be cut.

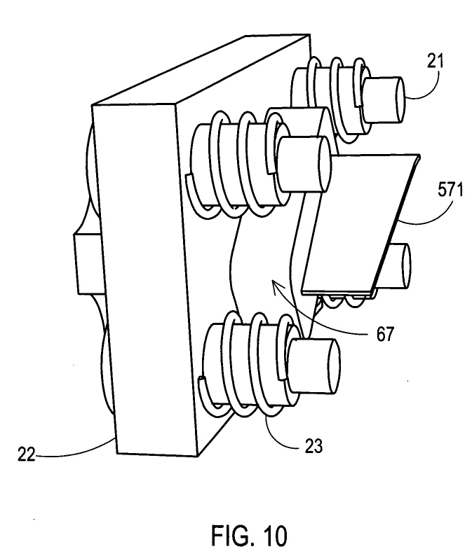

FIG. 10 provides a side view of an exemplary blade housing 22 suitable for use in the exemplary hand-held clamping and cutting device shown in FIG. 4. In this exemplary embodiment, blade housing 22 comprises blade holder 67, blade 571, shoulder screws 22, and compression springs 23. Shoulder screws 22 connect blade housing 22 to upper clamping member 19 (see FIG. 4), while compression springs 23 enable resistive movement of blade housing 22 and blade 571 toward upper clamping member 19 and an object to be cut (not shown). When a blade such as exemplary blade 571 is utilized, namely, a blade having a slanted blade edge (i.e., one end of the blade is positioned below an opposite end of the blade), lower clamping surface 181 of lower clamping member 18 (see, FIG. 3) may have a slot within lower clamping surface 181 capable of receiving a portion of blade 571 so that blade 571 can extend through an object to be cut.

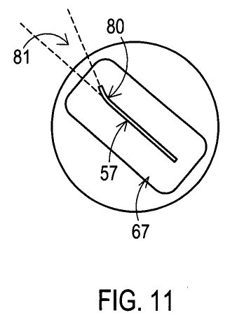

FIG. 11 provides a detailed view of exemplary blade holder 67 viewed from below blade holder 67. As shown in FIG. 11, exemplary blade holder 67 comprises blade 57 having a blade tip 80 that is bent at a blade tip angle 81 designated by the symbol a. Blade tip angle 81 typically ranges from about 1 to about 6020 , desirably from about 10 to about 45°, and more desirably about 30°. It had been discovered that having a bent or curved blade tip at one end of the blade (i.e., a blade tip angle 81 of about 30°) will cause, upon depression and cutting of a tubular graft, a resultant modified S-shaped curve on one end of the graft, which provides better attachment to an artery and subsequent flow through the arterial junction formed therefrom (see, for example, arterial junction 3 shown in FIG. 1).

### II. Methods of Making Clamping and Cutting Devices

The present invention is further directed to methods of making hand-held clamping and cutting devices. In one exemplary embodiment, the method of making a hand-held clamping and cutting device comprises providing an upper clamping member having an upper surface and an upper clamping surface, and an opening extending through the upper clamping member from the upper surface to the upper clamping surface; providing a lower clamping member having an lower clamping surface; connecting the lower clamping member to the upper clamping member so that the upper and lower clamping surfaces face one another and are operatively adapted to provide a clamping pressure on an object disposed therebetween; and providing a blade housing positioned above the upper clamping member, wherein the blade housing comprises a blade dimensioned so as to extend through the opening, the blade housing being operatively adapted to move toward the upper surface so that the blade extends through the opening and approaches the lower clamping surface.

Each of the device components may be formed using conventional techniques and materials. For example, the device components may be formed from materials such as polymeric materials, metallic materials, ceramic materials, or any combination thereof. Typically, device components such as upper and lower clamping members, upper and lower handles, blade housing, blade holder, and alignment members are formed from one or more polymeric materials (e.g., polyethylene or an acylonitrile butadiene styrene (ABS) copolymer), while device components such as the blade, any screws, any compression springs, and any torsion spring are formed from metallic materials (e.g., aluminum or stainless steel).

In one desired embodiment, the device is formed from a sterilizable material, such as an acrylonitrile-butadiene-styrene copolymer. In this embodiment, the device may be sterilized with, for example, a gamma radiation dose of at least about 25 kGy. In some embodiments, the device is disposable (i.e., used for a single time and then disposed of). In other embodiments, the device is reusable. If the device is reusable, the device is desirably thoroughly cleaned and/or sterilized after each use.

Suitable techniques for forming various components of the device include, but are not limited to, thermoforming techniques such as injection molding and extrusion, metal casting, metalworking (i.e., for blade formation), etc. Any conventional method of forming a polymeric or metal part may be used to form the various components of the disclosed clamping and cutting device of the present invention.

Device dimensions may vary as desired. In one desired embodiment, the device is dimensioned so as to be a hand-held device. Most operating rooms have limited space, so bulky machines are not practical for cutting a small (e.g., 6 or 8 mm diameter) prosthetic graft. Desirably, the device is large enough to cut a graft, but small enough so that a single person can operate the device using one or both hands.

Regarding the design and dimensions of suitable blades, computer modeling may be used to determine optimum design of a given blade and the cut angle for a particular application. For example, it has been determined that the blade design and cut angle can be optimized so as to create a cut graft that provides superior fluid flow through an arterial junction formed from the cut graft (see, for example, arterial junction 3 shown in FIG. 1).

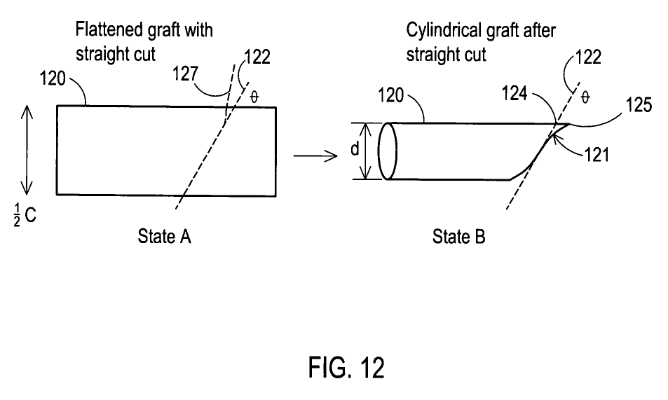

FIG. 12 provides a view of the result of cutting a tubular object (e.g., a synthetic graft) using the exemplary hand-held clamping and cutting device shown in FIG. 4. As shown in FIG. 12, when cutting a tubular synthetic graft 120 having a circumference C and a diameter d at a cut angle θ along a cut line 122 in a clamped, flattened state (i.e., State A), the resulting three-dimensional cut line cut 121 has an S-shaped curve once synthetic graft 120 is allowed to reopen (i.e., return to a tubular shape from a flattened state) as shown in State B. By modifying the cut angle θ and/or the configuration of cut line 122, the configuration of three-dimensional cut line cut 121 may be optimized for use in forming an arterial junction.

Further, with an understanding of how the cut object (e.g., a cut graft) is to be used, for example, in an arterial junction, further modifications may be made to the configuration of cut line 122 so as to optimize the configuration of three-dimensional cut line cut 121 for a particular application, such as for use in an arterial junction. Typically, a surgeon first sutures the heel (e.g., heel 5 shown in FIG. 1) of a graft (e.g., graft 1 shown in FIG. 1) to an artery (e.g., artery 2 shown in FIG. 1), then the toe (e.g., toe 6 shown in FIG. 1) of the graft, followed by the side portions of the graft to an artery to form an arterial junction (e.g., arterial junction 3 shown in FIG. 1). For such a surgical procedure, it has been determined that a modified cut line 127 using (i) a cut angle θ of about 40° and (ii) a blade tip angle 81 of about 30° (also referred to as angle a, see FIG. 11) results in a particularly desirable cut line 122 represented by a modified S-shaped curve in which a portion of tip 125 is removed from the toe-end of synthetic graft 120. In this exemplary embodiment, the modified S-shaped curve ends at a point along synthetic graft 120 away from tip 125 and close to point 124 shown in FIG. 12. The resulting synthetic graft 120 provides better attachment to an artery using the above-mentioned procedure, as well as improved flow through the arterial junction formed therefrom.

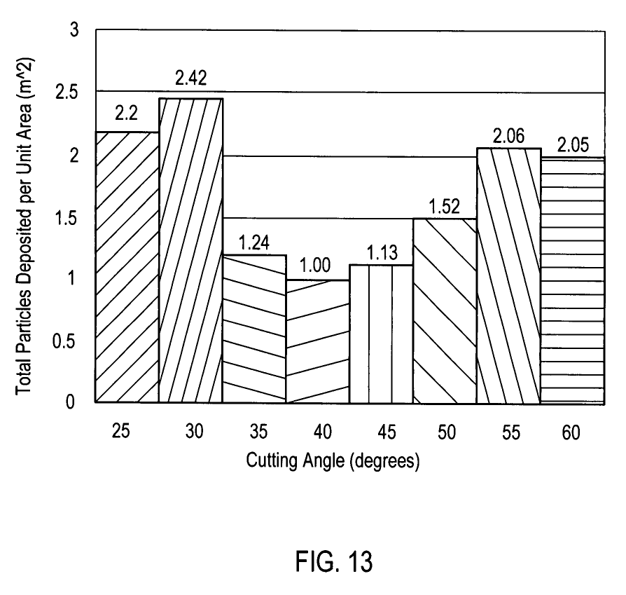

In one exemplary embodiment of the present invention, three-dimensional reconstructed arterial junctions were designed using CFX software and the dimensions of cut synthetic grafts formed using the above modified blade having one straight end and one curved end with a blade tip angle 81 of about 30°. Fluid flow velocity, particle deposition, and resident time within arterial junctions were measured while varying cut angle θ. Simulations utilized a cut angle θ ranging from 25° to 60°. It was determined that a cut angle θ of about 40° resulted in favorable fluid flow velocity, particle deposition, and resident time within an arterial junction. A cut angle θ of about 40° resulted in the least amount of deposited particles into the walls of the simulated anterior-graft junction as shown in FIG. 13. Further, a cut angle θ of about 40° resulted in the least amount of resident time for a given volume of blood to flow through an arterial junction of an artery at 2.185 seconds (within a range of 2.185 to 9.32 seconds).

### III. Methods of Using Clamping and Cutting Devices

The present invention is even further directed to methods of using hand-held clamping and cutting devices. In one exemplary embodiment, the method of using a hand-held clamping and cutting device comprises cutting an object such as a synthetic graft. The method of cutting an object may comprise (a) placing the object between upper and lower clamping surfaces of a clamping and cutting device, wherein the clamping and cutting device comprises (i) an upper clamping member having an upper surface and the upper clamping surface, and an opening extending through the upper clamping member from the upper surface to the upper clamping surface; (ii) a lower clamping member having the lower clamping surface, wherein the upper and lower clamping members are operatively adapted to provide a clamping pressure on the object; (iii) a blade housing positioned above the upper clamping member; and (iv) a blade connected to the blade housing, wherein the blade housing is operatively adapted to move toward the upper surface so that the blade extends through the opening and contacts the lower clamping surface; and (b) moving the blade housing toward the object so that the blade contacts and cuts the object.

In one desired embodiment, the method of cutting an object comprises placing the object between the upper and lower clamping surfaces of the above-described clamping and cutting device, and moving the blade housing toward the object so that the blade contacts and cuts the object. The method is particularly useful when the object comprises a graft, such as a synthetic graft.

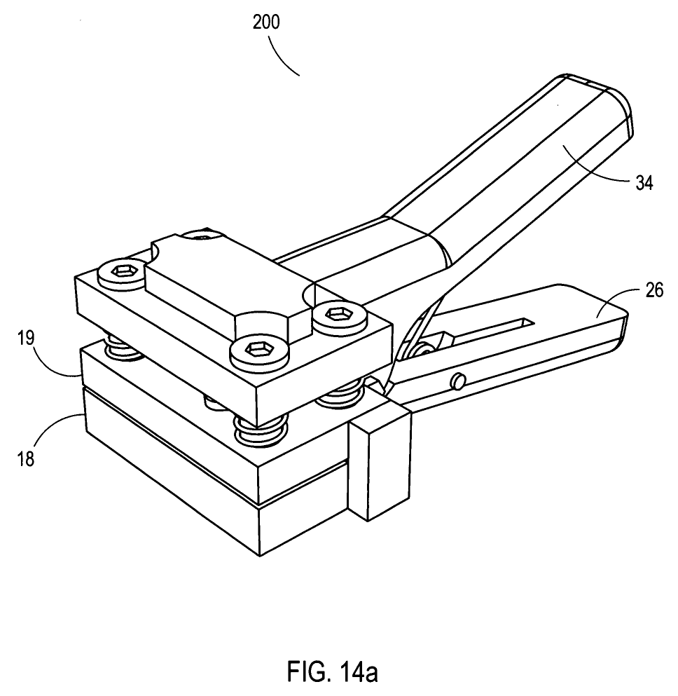

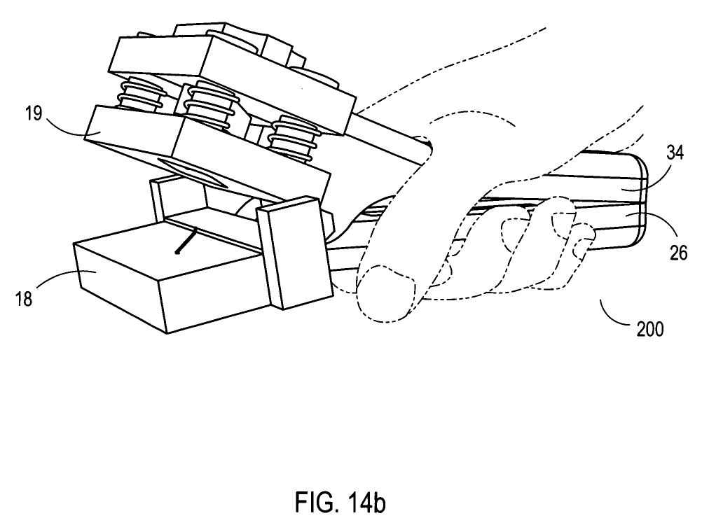

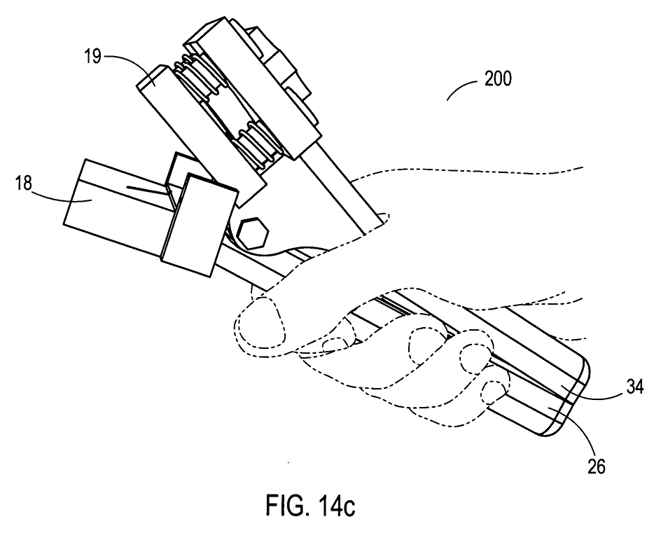

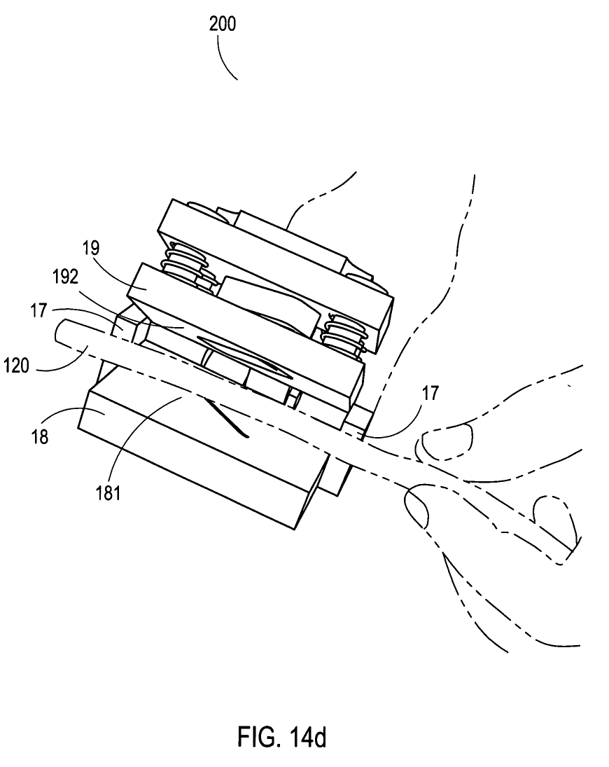

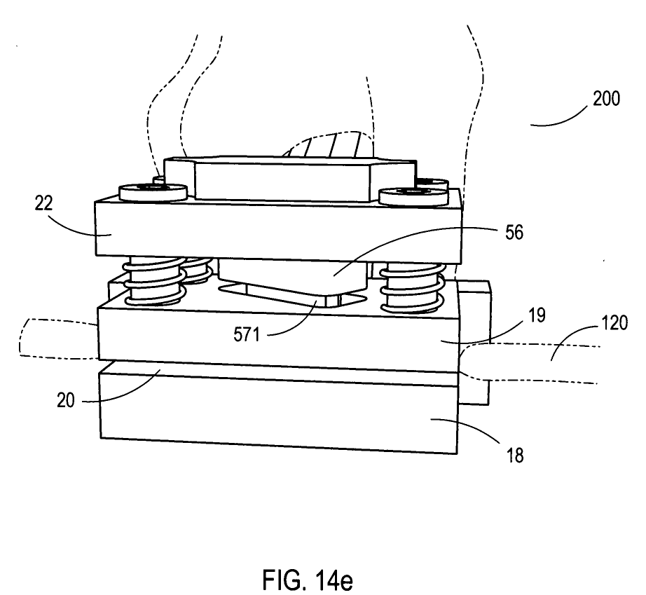

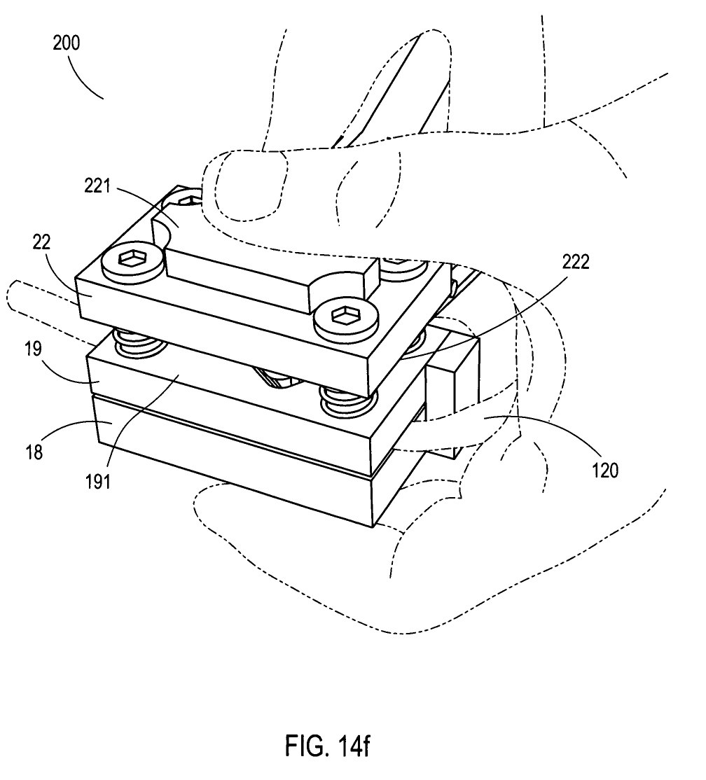

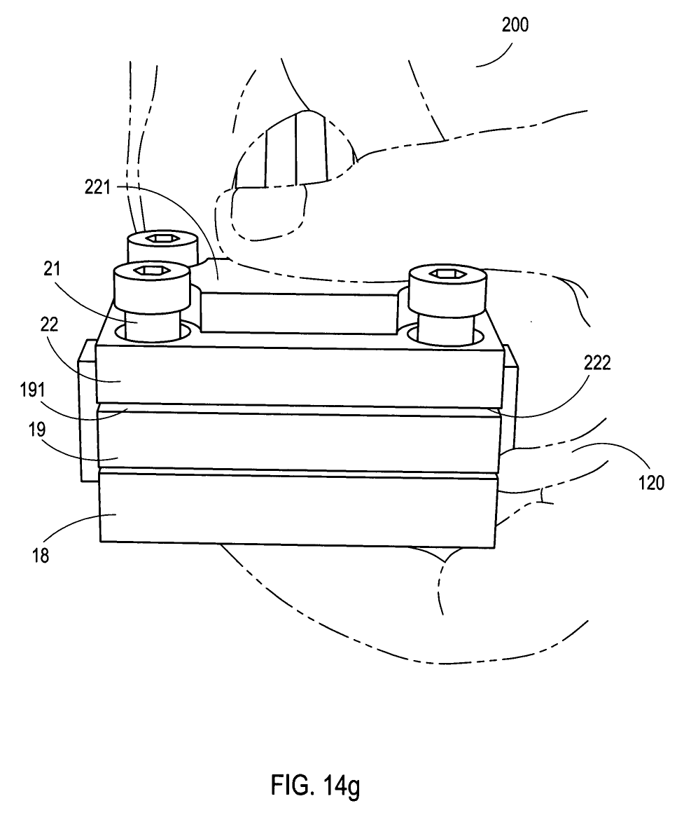

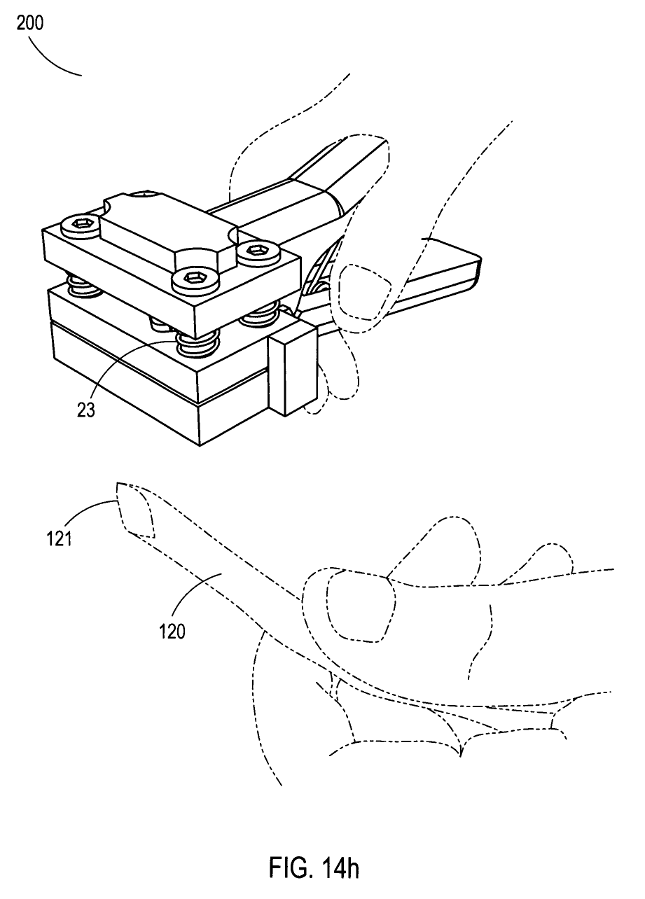

FIGS. 14a-14h depict exemplary process steps of cutting a synthetic graft using the exemplary hand-held clamping and cutting device shown in FIG. 4. As shown in FIG. 14a, exemplary hand-held clamping and cutting device 200 comprising upper clamping member 19, lower clamping member 18, upper handle 34 and lower handle 26 is in a relaxed state. In FIG. 14b, upper handle 34 is forced toward lower handle 26, which results in upper clamping member 19 moving away from lower clamping member 18. At this point, exemplary hand-held clamping and cutting device 200 is in an “opened” position. In FIG. 14c, exemplary hand-held clamping and cutting device 200 in an “opened” position is tilted upward to form a ledge onto which can be placed an object to be cut.

In FIG. 14d, a synthetic graft 120 is placed between upper clamping surface 192 of upper clamping member 19 and lower clamping surface 181 of lower clamping member 18 along lower clamping surface 181 so as to rest against alignment members 17 positioned along opposite sides of lower clamping member 18. In FIG. 14e, pressure is released from upper and lower handles 34,26 to provide a clamping force onto synthetic graft 120 by upper clamping surface 192 of upper clamping member 19 and lower clamping surface 181 of lower clamping member 18. As shown in FIG. 14e, synthetic graft 120 is securely positioned within gap 20 and below blade housing 22, blade holder 56, and blade 571.

In FIG. 14f, blade housing 22 with blade holder 56, and blade 571 is positioned in a relaxed state above upper clamping member 19 so that lower surface 222 of blade housing 22 is positioned above and away from upper surface 191 of upper clamping member 19. As shown in FIG. 14f, pressure is about to be applied to upper surface 221 of blade housing 22. In FIG. 14g, pressure has been applied to upper surface 221 of blade housing 22 so as to force blade holder 56, and blade 571 downward so as to contact and cut synthetic graft 120 until lower surface 222 of blade housing 22 comes into contact with upper surface 191 of upper clamping member 19. As shown in FIG. 14g, shoulder screws 21 are exposed due to compression of compression springs 23 between blade housing 22 and upper clamping member 19.

In FIG. 14g, blade housing 22 with blade holder 56 and blade 571 is allowed to return to a relaxed state (exposing compression springs 23 between blade housing 22 and upper clamping member 19). Further, upper and lower handles 34,26 are also allowed to return to a relaxed state so as to remove any clamping pressure from cut synthetic graft 120. Cut synthetic graft 120 having three-dimensional cut edge 121 is then removed from gap 20.

In some embodiments of the present invention, the method of using a hand-held clamping and cutting device comprises utilizing the hand-held clamping and cutting device in the form of a kit with one or more other kit components. In one exemplary embodiment, the kit comprises a clamping and cutting device as described above, and (i) a set of blades having two or more differing blade configurations, (ii) one or more synthetic grafts (i.e., uncut graft materials), or both (i) and (ii).

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A hand-held clamping and cutting device comprising:
an upper clamping member having an upper surface and an upper clamping surface, and an opening extending through the upper clamping member from the upper surface to the upper clamping surface;
a lower clamping member having an lower clamping surface, wherein the upper and lower clamping members are operatively adapted to provide a clamping pressure on an object disposed therebetween;
a blade housing positioned above the upper clamping member; and
a blade connected to the blade housing;
wherein the blade housing is operatively adapted to move toward the upper surface so that the blade extends through the opening and approaches the lower clamping surface.

2. The clamping and cutting device of claim 1, wherein the upper clamping surface is separated from the lower clamping surface by a gap having a gap thickness.

3. The clamping and cutting device of claim 2, wherein the gap thickness is up to about 1 cm.

4. The clamping and cutting device of claim 2, wherein the gap thickness is up to about 0.5 cm.

5. The clamping and cutting device of claim 1, wherein the upper and lower clamping members are connected to upper and lower handles respectively, said upper and lower handles being operatively adapted to move the upper clamping surface relative to the lower clamping surface.

6. The clamping and cutting device of claim 1, wherein the upper and lower handles are connected to one another such that when the upper and lower handles are moved toward one another, the upper clamping surface moves away from the lower clamping surface.

7. The clamping and cutting device of claim 1, wherein the upper and lower clamping surfaces are substantially planar, and when the device is at rest, the upper and lower clamping surfaces are substantially parallel with one another.

8. The clamping and cutting device of claim 1, further comprising a spring mechanism operatively adapted so as to resist movement of the upper and lower handles toward one another.

9. The clamping and cutting device of claim 1, wherein the blade housing is separated from the upper surface by at least one compression spring, such that upon applying suitable pressure to the blade housing, the compression spring will depress and the blade will pass through the opening and contact or extend into the lower clamping surface.

10. The clamping and cutting device of claim 1, wherein the blade housing is separated from the upper surface by four compression springs positioned along an outer periphery of the upper clamping member.

11. The clamping and cutting device of claim 1, wherein the blade is removably connected to the blade housing.

12. The clamping and cutting device of claim 1, further comprising one or more alignment members extending along the lower clamping member, said one or more alignment members being operatively adapted to align an object on the lower clamping surface in a first direction.

13. The clamping and cutting device of claim 12, further comprising a pair of alignment members extending along opposite sides of the lower clamping member toward the upper clamping member.

14. The clamping and cutting device of claim 12, wherein the blade is oriented at approximately a 40° angle from a longitudinal axis extending along the first direction.

15. The clamping and cutting device of claim 14, wherein the blade has one straight end and an opposite curved end.

16. The clamping and cutting device of claim 15, wherein the blade contains a bent therein having a bend angle of about 30°.

17. The clamping and cutting device of claim 1, wherein the upper and lower clamping members comprise a polymeric material.

18. The clamping and cutting device of claim 17, wherein the polymeric material is sterilizable.

19. The clamping and cutting device of claim 17, wherein the polymeric material is polyethylene or an acylonitrile butadiene styrene (ABS) copolymer.

20. The clamping and cutting device of claim 1, wherein the device is disposable.

21. A kit comprising:
the clamping and cutting device of claim 1; and
a set of blades having two or more differing blade configurations.

22. The kit of claim 21, further comprising:
one or more synthetic grafts.

23. A method of cutting an object comprising:
placing the object between the upper and lower clamping surfaces of the clamping and cutting device of claim 1;
moving the blade housing toward the object so that the blade contacts and cuts the object.

24. The method of claim 23, wherein the object comprises a graft.

25. A method of making a hand-held clamping and cutting device, said method comprising:
providing an upper clamping member having an upper surface and an upper clamping surface, and an opening extending through the upper clamping member from the upper surface to the upper clamping surface;
providing a lower clamping member having a lower clamping surface;
connecting the lower clamping member to the upper clamping member so that the upper and lower clamping surfaces face one another and are operatively adapted to provide a clamping pressure on an object disposed therebetween; and
providing a blade housing positioned above the upper clamping member, wherein the blade housing comprises a blade dimensioned so as to extend through the 20 opening, the blade housing being operatively adapted to move toward the upper surface so that the blade extends through the opening and approaches the lower clamping surface.

26. A method of cutting an object, said method comprising:
placing the object between upper and lower clamping surfaces of a clamping and cutting device, the clamping and cutting device comprising:
an upper clamping member having an upper surface and the upper clamping surface, and an opening extending through the upper clamping member from the upper surface to the upper clamping surface;
a lower clamping member having the lower clamping surface, wherein the upper and lower clamping members are operatively adapted to provide a clamping pressure on the object;
a blade housing positioned above the upper clamping member; and
a blade connected to the blade housing, wherein the blade housing is operatively adapted to move toward the upper surface so that the blade extends through the opening and approaches the lower clamping surface; and
moving the blade housing toward the object so that the blade contacts and cuts the object.

27. The method of claim 26, wherein the object comprises a graft.
